(19) United States
(12) Patent Application Publication
(10) Pub. No.: US 2005/0238536 A1
(43) Pub. Date: Oct. 27, 2005

(54) Device and method for measuring glycosaminoglycans in body fluids

(76) Inventors: Steven K. Striepeke, Sebastopol, CA (US); Zhiqi Gouillon, Petaluma, CA (US)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 10/828,780

(22) Filed: Apr. 21, 2004

(51) Int. Cl. G01N 15/06

(52) U.S. Cl. 422/68.1; 436/94

(57) ABSTRACT

The present invention features automated devices, kits, and assay methods for measuring glycosaminoglycan levels in body fluids such as urinary glycosaminoglycan excretion or blood heparin levels. Such devices, kits and methods are useful, e.g., for monitoring heparin therapy or diagnosing or monitoring mucopolysaccharidoses.

### FIELD OF THE INVENTION

The present invention is in the field of biochemistry and medical and laboratory devices. The present invention provides a device and methods useful for detecting and quantifying glycosaminoglycans.

### BACKGROUND OF THE INVENTION

Carbohydrates play a number of important roles in the functioning of living organisms. In addition to their metabolic roles, carbohydrates are structural components of the human body covalently attached to numerous other entities such as proteins and lipids (called glycoconjugates). For example, the human endothelium cell surface makeup includes a glycoprotein matrix. The carbohydrate portion of this matrix imparts important properties to the endothelial cell surface and therefore internal blood vessel structure and the fluidity of the blood that interacts with the endothelium surface.

Glycosaminoglycans are sugar chains consisting of repeating polymers of acidic polysaccharides. These materials are composed of building blocks of the following sugars in various combinations: galactose, glucose, N-acetylglucosamine, N-acetylgalactosamine, glucuronic acid, galacturonic acid, and iduronic acid. In addition these sugar units may be variably linked α or β at their anomeric carbons and (1-3) or (1-4) to their ring carbons through an O-glycosidic bond. Finally, they may be variably substituted with sulfates at their 2, 3, 4, or 6 carbons. Depending on the precise repeating disaccharide structure and location of sulfates, human connective tissue glycosaminoglycans are commonly classified as chondroitin sulfates, dermatan sulfates, heparan sulfates, heparin sulfates, and keratan sulfates (Collins P M, Carbohydrates, London, Chapman Hall, 1987). Glycosaminoglycans are carbohydrates, which are integrally associated with endothelium and are thought to be the major source of naturally occurring anticoagulants in human blood. The biochemical nature of glycosaminoglycans is a variably N- and O-sulfated polymer of disaccharides of (heparin) [-glucuronic acid (1-4)-N-acetylglucosamine-(1-4)-]ₙ; and [-iduronic acid (1-4)-N-acetylglucosamine-(1-4)-)ₙ; (chondroitin sulfates) [-glucuronic acid (1-3)-N-acetylgalactosamine-(1-4)-]ₙ or (keratan sulfate) N-acetylglucosamine(1-3)-galactose-(1-4)-]ₙ (Casu, “Structure of Heparin and Heparin fragments, in Heparin and Related Polysaccharides, Structure and Activities,” Cohen et al. (eds), Ann NY Acad Sci 556:1-17, (1989)).

Glycosaminoglycans (GAGs) are present in mammalian blood, urine and other body fluids and are sensitive markers for the diagnosis of lysosomal storage diseases known as mucopolysaccharidoses (MPS) (Klock et al., Internat Pediatr. 9:40-48 (1994); Starr et al, Glycosylation & Disease 1:165-176 (1994)). They differ in clinical features, accumulated storage materials, and deficient enzyme or combination of enzymes. These lysosomal storage diseases are characterized by intralysosomal accumulation of undegraded glycosaminoglycans, excessive urinary excretion of glycosaminoglycans, progressive mental and physical deterioration, and premature death. Patients are usually born without the visible clinical features of MPS, but develop progressive clinical involvement. Each type of MPS has specific lysosomal enzyme deficiency with a characteristic degree of organ involvement and rate of deterioration. See Muenzer, Adv. Pediatri. 33:269-302 (1986). An overall increase in glycosaminoglycan excretion is indicative of a lysosomal storage disease, and identification of the type of glycosaminoglycans excreted, e.g. heparan sulfate, keratan sulfate, dermatan sulfate, and chondroitin-6-sulfate can be a specific marker to identify the type of disease.

In addition, the degradation products of glycosaminoglycans found in urine, and the secretion rates of specific glycosaminoglycans, such as heparin sulfate, may provide valuable information regarding the imbalance between endogenous heparin production and the formation of atherosclerotic plaques. Heparin sulfate as measured by a fluorophore-assisted carbohydrate electrophoretic process (FACE) developed by Glyko, Inc. (Novato, Calif.) has been demonstrated and may be useful for determining the presence of low levels of endogenously made heparin (Mielke et al., Blood 84: 197A (10 SUPPL. 1) (1994). Elevated levels of glycosaminoglycans in the urine have been correlated with osteoporosis (Todorova, S., et al, Horm Metab Res. 1992 December; 24(12):585-7) and kidney disease (Koshiishi, et al, Arch Biochem Biophys. 2002 May 1;401(1):38-43). Methods for measuring levels of endogenous heparin and methods for assessing risk for and monitoring the progress of development of atherosclerosis by determining the amount of endogenous heparin present in a mammal are disclosed in U.S. Pat. No. 6,291,439, the disclosure of which is herein incorporated by reference.

Typically known methods for the quantitative measurement and characterization of MPS are manual, complicated, and labor intensive. Other more rapid tests for screening may be subject to inaccuracies leading to false positives or false negatives.

Because of the high degree of sulfation of glycosaminoglycans, dyes which bind these sulfate groups have been used to detect heparin and other glycosaminoglycans. One method involves the purification of glycosaminoglycans by precipitation or other means, followed by complexing the glycosaminoglycans with dyes. The interaction of glycosaminoglycans with the dyes causes a shift in the peak absorbance of the dyes. These methods can be used to detect heparin and other glycosaminoglycans present in a sample. Disclosure of such methods is found in Sommer et al., U.S. Pat. No. 4,543,335; Karkar, U.S. Pat. No. 4,911,549; Yen et al, Biochem. Biophys. Acta. 184:646-648 (1969); Famdale et al., Biochimica et Biophysica Acta 883: 173-177 (1986); and Endobakhare et al., Analytical Biochemistry 243: 189-191 (1996). Additional methods featuring fluorophore-assisted carbohydrate electrophoresis (FACE) are described in U.S. Pat. Nos. 4,975,165, 5,035,786, 5,104,508, 5,019,231, 5,205,917, 5,316,638, 5,340,453, 5,472,582, and 5,087,337, the disclosures of which are incorporated herein by reference. However, none of these methods provide for an automated procedure for glycosaminoglycan analysis.

Whitley, U.S. Pat. No. 5,310,646 proposed automation of a method for detecting g lycosaminoglycans in a urine sample that has been dried on a paper matrix, which involves agitating the paper with water to extract the glycosaminoglycans, adding 1,9-dimethylmethylene blue chloride dye, and assessing the color change with a spectrometer. See also Whitley et al., Mol. Genet. Metab., 75:56-64 (2002).

Heparin is used as an anticoagulant in a variety of situations, including bypass surgery, post-operative antithrombotic therapy, deep vein thrombosis, pulmonary embolism, and recurrent thromboembolism. It is often co-administered with antithrombotics and thrombolytics such as tissue plasminogen activator (Majerus et al., “Anticoagulant thrombolytic and antiplatelet drugs” The Pharmacological Basis of Therapeutics, 9^{th} ed., Hardman et al. eds, McGraw Hill, (1996)). Heparin is the most commonly used anticoagulant, but suffers from the necessity of constant monitoring. Heparin levels are typically not measured directly. Rather, physicians depend on indirect measures such as clotting time or aPTT tests. Heparin has a narrow therapeutic index (Cipolle et al., in Advanced Pharmacokinetics, 3^{rd} ed. Evans, et al., eds, (1992)) and shows unusual pharmacokinetics that are highly variable between individuals (Bjornsson T D., J Pharm Sci 71: 1186 (1982); Kandrotas et al., Clin. Pharmacokin. 22: 359 (1992)). These studies show that heparin clearance is dose and context dependent with higher doses producing unique, triphasic kinetics. Since the consequences of under or over dosing heparin are serious, a simple, direct, and automated method of monitoring heparin concentrations in blood would be of great benefit.

It is an object of the present invention to provide a device, processor and method suitable for rapid and accurate detection of glycosaminoglycans over a wide range of concentrations and in a variety of body fluid samples.

### SUMMARY OF THE INVENTION

A first aspect of the invention provides a glycosaminoglycan measuring device comprising (1) a glycosaminoglycan separation cartridge, preferably an ion exchange resin, that is adapted to separate glycosaminoglycans from interfering substances in the sample; and (2) a detection apparatus, preferably a spectrophotometer, comprising a detection chamber coupled to the glycosaminoglycan separation cartridge, that is adapted to detect the separated glycosaminoglycans. The device may also include a sample entry port coupled to the glycosaminoglycan separation cartridge and adapted to accept a bodily fluid sample. The device may further include a reagent storage device coupled to the glycosaminoglycan separation cartridge and adapted to store reagents for delivery to the glycosaminoglycan separation cartridge. Optionally additional elements include (1) a first pump coupled to the glycosaminoglycan separation cartridge and adapted to pump fluids into the separation cartridge, and/or (2) a second pump coupled to the detection apparatus and adapted to pump fluids to the detection chamber, and/or (3) a controller adapted to control the operation of the first and second pumps, and/or (4) a user interface device operably connected to the detection apparatus and/or (5) a temperature controller operably connected to the detection apparatus and/or (6) a first fluid passageway coupled between the sample entry port and the glycosaminoglycan separation cartridge and adapted to deliver the sample from the sample entry port to the glycosaminoglycan separation cartridge; and/or (7) a second fluid passageway coupled between the glycosaminoglycan separation cartridge and the detection chamber and adapted to deliver the separated glycosaminoglycans from the glycosaminoglycan separation cartridge to the detection chamber and/or (8) one or more valves adapted to control liquid flow in one or more of the fluid passageways.

The controller may include a processor, a computer readable memory, and a routine stored on the computer readable memory and adapted to be executed on the processor to control the operation of the first and second pumps and/or other elements of the device.

The user interface device may include a user interface, a processor, a computer readable memory and a routine stored on the computer readable memory and adapted to be executed on the processor to analyze the output of the detection apparatus and display results of analysis to a user via the user interface. The user interface may be adapted to prompt a user to log in to the device, initialize the device, enter patient information, prime the device, start the routine, clean the device, reset a cycle counter, enter comments, enter known standard solutions, calculate standard curves, calculate amounts of glycosaminoglycans based on a standard curve, record user actions, and/or shutdown the device. Preferably the device utilizes software designed to quantify the amount of glycosaminoglycans present based on detecting the detection reagent bound to the separated glycosaminoglycans.

In one embodiment, the glycosaminoglycan measuring device comprises (1) a sample entry port coupled to a first transport line; (2) a first reagent container coupled to a second transport line; (3) a second reagent container coupled to a third transport line; (4) a glycosaminoglycan separation cartridge coupled to one or more transport lines; (5) a waste discharging port coupled to a fourth transport line; (6) a detecting reagent container coupled to a fifth transport line; (7) a pump operably connected to one or more of the transport lines; and (8) a detection apparatus operably connected to the device. Such a device may optionally include a reagent measuring apparatus in communication with one or more transport lines, and/or a gas entry port coupled to one or more transport lines, and/or a cleaning solution container coupled to a sixth transport line, and/or a transport line adapted to mix liquids, and/or a temperature controller operably connected to the detection apparatus, and/or a valve coupled to one or more transport lines and adapted to direct liquid flow.

In a related aspect, the invention provides a glycosaminoglycan measuring device comprising a sample entry port, separator means for substantially separating glycosaminoglycans from a sample delivered at the sample entry port to produce separated glycosaminoglycans, and detection means for detecting the separated glycosaminoglycans.

In a second aspect, the invention provides methods of measuring glycosaminoglycans in a body fluid sample, comprising the steps of: (a) automatically delivering a portion of the sample to a glycosaminoglycan separation cartridge; (b) separating glycosaminoglycans from interfering substances using the glycosaminoglycan separation cartridge; (c) automatically delivering the separated glycosaminoglycans to a detection apparatus; (d) combining a detection reagent with the separated glycosaminoglycans; and (e) detecting the amount of separated glycosaminoglycans by detecting the amount of detection reagent bound to the separated glycosaminoglycans. Preferably the automatic delivery occurs in response to user input.

Preferably the detection reagent is a metachromic dye that specifically binds glycosaminoglycans, most preferably dimethylmethylene blue (DMMB). Where the dye is DMMB, preferably the detection step includes shining light at a wavelength of about 526 nm+/−5 nm at the separated glycosaminoglycans and optionally further includes shining light at a wavelength of about 592 nm+/−5 nm at the separated glycosaminoglycans.

The methods of the invention may optionally include additional steps, including (1) analyzing an output of the detection apparatus and/or (2) displaying the analyzed output to a user and/or (3) regulating the temperature of the glycosaminoglycan-detection reagent complex. The step of separating glycosaminoglycans from interfering substances may comprise steps of binding glycosaminoglycans to a solid phase; removing interfering substances; and eluting glycosaminoglycans from the solid phase. In an embodiment where the solid phase within the separation cartridge is a cationic ion exchange resin, the step of removing interfering substances preferably includes contacting the solid phase with a buffered solution comprising salt (e.g., Na+) at a concentration ranging from 350 mM to 450 mM, most preferably 400 mM, and the step of eluting glycosaminoglycans preferably includes contacting the solid phase with a buffered solution comprising salt at a concentration ranging from 700 mM to 1500 mM, most preferably 1000-1200 mM.

Exemplary body fluid samples that may be analyzed according to the present invention include whole blood, plasma, serum, urine, cerebrospinal fluid, pleural fluid, extracts of tissue biopsies, saliva, semen, stool, sputum, tears, or mucus, preferably blood or urine. Exemplary glycosaminoglycans that may be detected according to the invention include chondroitin sulfates, dermatan sulfates, keratan sulfates, heparan sulfates, heparin sulfates, and/or heparin.

In a related embodiment, the method of the invention may be used to quantify specific types of glycosaminoglycans by adding one or more glycosaminoglycan-specific degrading enzymes to an aliquot of the sample before the sample is analyzed for glycosaminoglycan content. Exemplary glycosaminoglycan-specific degrading enzymes include chondroitinase B, chondroitinase AB, heparinase, heparitanase, heparitanase I, heparitanase II, keratanase, α-L-iduronidase, iduronate sulfatase, Heparan N-sulfatase, N-acetylglucosaminidase, α-glucosamine-N-acetyltransferase, α-glucosamine-6-sulfatase, N-acetylgalactosamine-6-sulfatase, B-galactosidase, N-acetylgalactosamine-4-sulfatase, or B-glucuronidase.

In a third aspect, the invention provides kits for use in the device of the invention comprising instructions for using a device of the invention and one or more reagents for use in such a device. Other kits may include one or more standards of known glycosaminoglycan concentration.

The methods, devices and kits of the present invention may be used to detect GAG levels, for example, for monitoring heparin therapy, for measuring endogenous heparin levels, for measuring urinary GAGs to screen newborns, infants, or children for disorders such as mucopolysaccharidoses associated with abnormal GAG levels, or for monitoring response of patients with such disorders to therapy.

These and other aspects and features of the invention will become more apparent from the following detailed description when taken in conjunction with the accompanying drawings.

### BRIEF DESCRIPTION OF THE FIGURES

FIG. 1 is a schematic representation of the elements of an illustrative automated glycosaminoglycan analyzer device according to the present invention.

FIG. 2 is a second schematic representation of the elements of another illustrative automated glycosaminoglycan analyzer device according to the present invention.

FIG. 3 is a schematic representation of a hardware and operating system which could be used to operate a device according to the present invention.

FIG. 4 is a graphical representation of data showing measurements of heparin level in units per milliliter for a patient plasma sample over time using the device and methods according to the present invention (represented by the thinner line), as well as measurements of activated clotting time (ACT) for the same sample (represented by the thicker line).

FIG. 5 is a graphical representation of data showing correlation of manual vs. automated device measurements of glycosaminoglycan (GAG) levels for urine samples of patients suffering from mucopolysaccharidosis I, by using the manual DMMB dye-binding method and by using the device and methods according to the present invention. The measurements using the manual method are plotted on the X-axis and measurements using the automated device are plotted on the Y-axis.

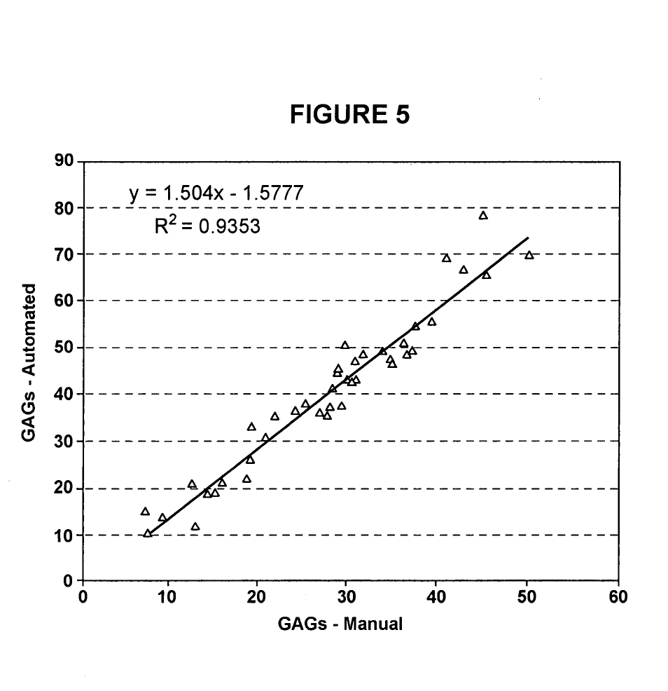

While the disclosure is susceptible to various modifications and alternative constructions, certain illustrative embodiments thereof have been shown in the drawings and will be described below in detail. It should be understood, however, that there is no intention to limit the disclosure to the specific forms disclosed, but on the contrary, the intention is to cover all modifications, alternative constructions, and equivalents falling within the spirit and scope of the disclosure as defined by the appended claims.

### DETAILED DESCRIPTION OF THE INVENTION

One aspect of the invention provides an automated glycosaminoglycan detection device that provides an automated, direct biochemical measurement of glycosaminoglycans.

In one exemplary embodiment, the device quantifies the total sulfated glycosaminoglycans in urine, normalized to the concentration of creatinine in urine. The device relies on the spectrophotometric detection at a wavelength of 592 nm of metachromatic changes in 1,9-dimethylmethylene-blue (DMMB) dye that occur when the glycosaminoglycans complex with DMMB. The device is calibrated with a set of standards, and then urine samples or controls are manually loaded on the machine. During the automated assay process, urinary glycosaminoglycans are separated from interfering substances using a cationic ion-exchange column. The sample is taken up by the device, diluted with a sample loading solution, and loaded onto the ion exchange column. The column is washed with a column wash solution. The glycosaminoglycans are eluted from the column with an eluent, mixed with acidified DMMB, and detected by measuring absorbance of 592 nm light. From the raw absorbance data and sample loading volume, the instrument software calculates mg/mL concentration values for total glycosaminoglycans using a standard curve calculated using absorbance data of the glycosaminoglycan standards.

One of ordinary skill in the art can easily vary solutions to suit analytes or other circumstances of implementation. For example, the sample loading solution may include 150 mM to 225 mM NaCl, pH 7.2-8.8, the column wash solution may include 350 mM to 450 mM NaCl, pH 7.2-8.8, the eluent may include 700 mM-1500 mM NaCl, pH 7.2-8.8. Alternative salts, such as KCl, are also possible. In one embodiment of the device, the eluent is 1200 mM NaCl, and in another embodiment of the device the eluent is 1000 mM NaCl.

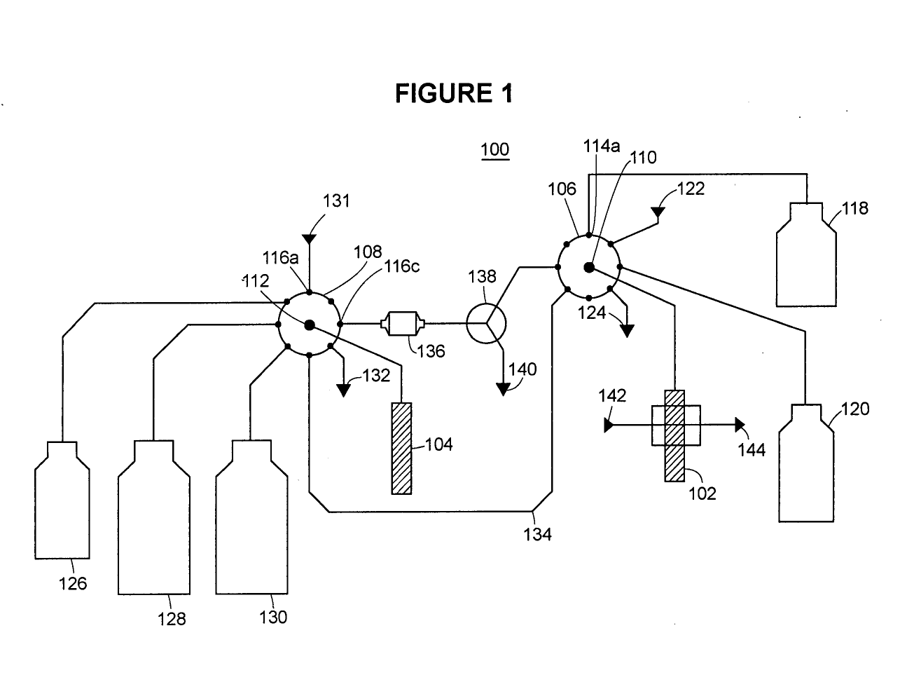

FIG. 1 represents a schematic representation 100 of the elements of an exemplary automated glycosaminoglycan analyzer device according to the present invention. A sample entry port 131 is provided for introducing a body fluid sample and may be operably connected to a sample port bay to which the operator has access. The sample port 131 may also be operably connected to one or more reagent containers such as a diluent container 128 and/or salt solution containers 126 and/or 130. The analyzer takes a designated volume of body fluid from a sample tube, e.g., 0.05 mL, 0.2 mL, or 1.5 mL, then mixes it with an appropriate buffered isotonic solution (“sample loading solution”, e.g., 200 mM sodium chloride, 20 mM trizma base). The body fluid sample and the buffered isotonic solution may be mixed in a vial that the operator places in a sample port bay. The mixing of the body fluid sample with the buffered isotonic solution may be performed by alternately aspirating and delivering the sample-sample loading solution mixture between the sample vial and the second syringe pump 104. The volume sampled may be preset, or determined based on physiological values such as urine creatinine levels. Those of skill in the art may easily optimize the amount of the sample according to the physiological conditions without undue experimentation. A port 132 is also provided for waste removal. In operation, the second rotary valve 108 is set to position 116a and a sample to be tested (not depicted) is introduced into the sample port 131 by activating the second syringe pump 104.

The system 100 uses a first and second syringe pump 102, 104. The pumps 102, 104 allow dispensing precise amounts of fluid. The syringe pumps 102, 104 are coupled to rotary valves 106, 108 respectively. Also included in these pump assemblies is a separate linear actuator for the syringe. The rotary valves 106, 108 allow a common port 110, 112 coupled to the syringe pumps 102, 104 to be connected to any of the respective peripheral eight ports 114a-h, 116a-h. A central port 110 links one peripheral port to the syringe at any point in time. A controller 300 controls both the syringe pumps 102, 104 and the rotary valves 106, 108. In an exemplary embodiment, the first syringe pump 102 is coupled through the first rotary valve 106 to a plurality of fluid tanks 118, 120 holding, for example, an ethanol wash and DMMB dye solution or other detection reagent (“Indicator”). Other ports 114b, 114d may be coupled to first rotary valve 106 may be further coupled to a diluent port 122 and/or a waste outlet 124. The diluent introduced at this port 122 may be used for washing of syringes and fluidics and may also be mixed with a salt solution to make a sample loading solution that is used to dilute the sample. The diluent may be, for example, a 20 mM trizma base having a pH around 8.3.

Similarly, the second rotary valve is coupled to fluid tanks 126, 128, 130. In an exemplary embodiment, the tank 126 contains a buffered intermediate concentration salt solution (“Buffer”), tank 128 contains Diluent or buffered salt solution for mixing with the body fluid sample, and tank 130 contains buffered high concentration salt solution (“Eluent”). Other ports 116a, 116d may be coupled to a sample inlet 132 and a second waste outlet 134. A mixing line 134 is coupled between the rotary valves 106, 108 at ports 114f and 116e respectively.

To eliminate the need for yet another tank that contains the sample loading solution, online mixing of Diluent and Buffer is employed to prepare the sample loading solution, which is then mixed with the sample for loading onto the column. For example, a sample loading solution of 200 mM NaCl is prepared by mixing Diluent (0 mM salt) with Buffer (400 mM salt) in equal amounts. Diluent and Buffer are drawn into second syringe pump 104 in small, equal, alternating volumes and expelled into the sample vial in the sample bay thereby delivering a net 200 mM solution for diluting the sample. That mixture is drawn up into second syringe pump 104 and loaded onto the column.

Alternatively, tank 126 or 130 could be eliminated if online mixing of Diluent with a high salt solution is used to prepare not only the sample loading solution but also the Buffer and Eluent. In such an embodiment, only two solutions would be needed, a high-salt solution and a diluent, both containing the same buffer concentration and both adjusted to the same pH.

In order to make an accurate colorimetric glycosaminoglycan measurement, cells, proteins, and other interfering substances are preferably removed from the body fluid sample. The device preferably automates the colorimetric measurement steps and the sample preparation steps (removal of cells and protein, separation of the glycosaminoglycan). In a preferred embodiment, the automated glycosaminoglycan analyzer incorporates ion exchange resins for the separation of glycosaminoglycans from samples, automated washing protocols to remove competing or interfering substances, and automated elution of glycosaminoglycans to a mixing chamber, or in-line reactor, for detection by dye binding.

Referring again to FIG. 1, a schematic representation 100 of analyzer of the present invention, a glycosaminoglycan capture cartridge 136 is provided for substantially separating glycosaminoglycans from the remaining components of the body fluid. The cartridge 136 is coupled at one end to port 116c. A solenoid valve 138 couples the cartridge 136 to a third waste outlet 140 for removing waste materials including the remaining components of the body fluid. The diluted sample is infused through an ion-exchange cartridge 136 in order to extract glycosaminoglycans from the sample solution. Under these conditions, some interfering proteins adsorb to the media in the ion-exchange cartridge. Cells and most interfering substances are separated from the glycosaminoglycan and sent to waste through a waste port 140. An appropriate ion exchange cartridge of suitable size and shape may be designed by skilled artisans without undue experimentation. Residual protein in the glycosaminoglycan capture cartridge maybe removed with a wash of column wash solution (buffered intermediate-concentration salt) from 126. Glycosaminoglycans are mobilized toward a colorimetric processor with eluent (buffered, high-concentration salt solution) from 130.

Sample Input Apparatus and Methods

Body fluid samples that can be analyzed for glycosaminoglycan content according to the devices and methods of the invention include, but are not limited to, whole blood, plasma, serum, urine, cerebrospinal fluid, pleural fluid, extracts of tissue biopsies, saliva, semen, stool, sputum, tears, mucus, and other biological fluids.

Exemplary glycosaminoglycans detectable according to the present invention include chondroitin sulfates, dermatan sulfates, keratan sulfates, heparan sulfates, heparin sulfates, and heparin. Glycosaminoglycans are polyanions and hence bind polycations and cations, such as Na⁺ and K⁺. This latter ability attracts water by osmotic pressure into the extracellular matrix and contributes to its turgor glycosaminoglycans also gel at relatively low concentrations. The long extended nature of the polysaccharide chains of glycosaminoglycans and their ability to gel, allow relatively free diffusion of small molecules, but restrict the passage of large macromolecules. Because of their extended structures and the huge macromolecular aggregates they often form, they occupy a large volume of the extracellular matrix relative to proteins. Murry R K and Keeley F W; Harper's Biochemistry, 24th Ed. Ch. 57. pp. 667-85.

Chondroitin sulfate and dermatan sulfate are both derived from the same polymer D-glucuronic acid beta (1-3)D-N-acetyl galactosamine beta (1-4). They can be sulfated at positions 4 or 6 of N-acetyl galactosamine. They are not N-sulfated. The difference between chontroitin sulfate and dermatan sulfate is the epimerisation of glucuronic acid to iduronic acid.

Chondroitin sulfate A (GlcUA-GalNAc-4S) is a historical alternative name for chondroitin 4-sulfate, i.e., chondroitin sulfate, which is sulfated on the C4 position of the GalNAc. Chondroitin sulfate B (IdoUA-GalNAc-4S) is a historical alternative name for dermatan sulfate. It is sulfated on the C4 position of GalNAc but the C5 of the uronic acid has undergone epimerisation to iduronic acid. Chondroitin sulfate C (GlcUA-GalNAc-6S) is a historical alternative name for chondroitin 6-sulfate, i.e., C6S, which is sulfated on the C6 position of the GalNAc. Chondroitinase AC and ABC cleave chondroitin sulfate, while chondroitinase B cleaves dermatan sulfate.

Keratan sulfate is a polymer based upon a repeating N-acetyllactosamine sequence. There are two repeating sugars are N-acetylglucosamine and galactose. Other sugars, notably fucose, are also present as branches along the main chain. The structure of a keratan sulfate chain can be considered as having three sections: a non-reducing terminal chain cap, a linkage to a protein core at the reducing terminal end of the chain, and a repeat region which connects the two. Enzymes that digest keratan sulfate include Keratanase (E.C 3.2.1.103) and Keratanase II (Bacillus sp.).

Heparin is a highly sulfated, long-chain polysaccharide of N-acetyl-glucosamine alternating with either glucuronic or iduronic acid with an average molecular weight in the unfractionated form (UFH) in excess of 15,000 daltons. The average molecular weight of low molecular weight heparin (LMWH) is approximately one-third that of UFH. Heparin functions as an anticoagulant by binding to and modulating blood serine proteases and activated clotting factors, primarily through the heparin-antithrombin III (ATIII) complex. A unique pentasaccharide responsible for the binding of heparin to ATIII is found only on one-third of heparin molecules (Hirsch, New Engl J Med 324:1565 (1991)). Both forms of heparin are capable of releasing tissue factor pathway inhibitor (TFPI), a natural anticoagulant found in the endothelial cells lining the blood vessels (Fareed et al. Clin Appl Thrombosis/Hemostasis 2(3):200-208 (1996)). The primary use of heparin is to treat thrombotic disorders such as deep venous thrombosis and as an anticoagulant during surgical procedures. Individual response to heparin is variable and its efficacy relies on titrating a dose of heparin that will prevent further clot formation without adverse side effects. Inadequate initial dosage with heparin can lead to thromboembolic events, while excess heparin dosage may cause bleeding, a major complication of therapy.

Heparin sulfate is a common material found in all mammalian connective tissues. It is a variably N- and O-sulfated polymer of disaccharides of glucuronic acid-α(1-4)-N-acetylglucosamine and iduronic acid α(1-4)-N-acetylglucosamine (Casu, “Structure of Heparin and Heparin Fragments”, in Heparin and Related Polysaccharides, Structure and Activities, Cohen, et al eds., Ann N.Y. Acad Sci 556:1-17 (1989)). Heparan is a polymer much like heparin but distinguished from it as being less sulphated and having more a cetyl contents. Heparan also has reduced iduronic acid content in favor of uronic acid, relative to heparin (Lindahl, U et al, Ann. Rev. Biochem., 1978, 47, 385, Kennedy, J. F., Proteoglycans-Biological and Chemical Aspects in Human Life, Elsevier publishers, 1979)

Heparinase degrades the most highly sulphated species, Heparitanase II will degrade heparin with relatively low sulphation and highly sulfated heparan while Heparitanase and Heparitanase I degrade the less sulphated heparan species (see http://www.seikagaku-hit.com/english/02tech/enz/04ko/p01.htm for a chart illustrating the substrate/enzyme continuum.

Mucopolysaccharidoses (MPS disorders) resulting from glycosidase enzyme deficiency are known to result in elevated excretion of urinary glycosaminoglycans (GAGs). The excess glycosaminoglycans can be expected to be terminated at their non-reducing terminal with the specific monomer that cannot be removed, in-vivo, in the absence of that enzyme. This inability to remove terminal monomers can result in the accumulation, and subsequent excess excretion of one or more of the various classes of glycosaminoglycans (e.g., Dermatan Sulfate, Heparan Sulfate, Keratan Sulfate, Chondroitin Sulfate).

A glycosaminoglycan analyzer device of the present invention can be employed to identify in a quantitative manner the type of glycosaminoglycans present in the sample. The device is used to measure total glycosaminoglycan content of a patient sample as well as total glycosaminoglycan content of a patient sample that has been depleted of a class or classes of glycosaminoglycans by specific enzymatic depolymerizations of target glycosaminoglycans. For example, an aliquot of the patient sample can be pre-treated with the deficient enzyme and/or with one or more enzymes specific for certain types of glycosaminoglycans. When glycosaminoglycan content of the patient sample is measured before and after treatment with the glycosaminoglycan-specific enzyme(s), the differential between the two values indicates how much of the glycosaminoglycan content was due to the glycosaminoglycan that was specifically degraded by the enzyme(s). For example, use of the suspected deficient enzyme on the patient glycosaminoglycan sample to remove the terminal monomer, will result in a polymer that can be enzymatically depolymerized or digested. Depolymerized glycosaminoglycans that have been reduced into small component oligosaccharide units (such as disaccharides) produce a negative response with certain embodiments of the glycosaminoglycan analyzer device of the present invention since the resultant fragments will be too small to be retained through the GAG extraction process on the ion-exchange media or because its interaction with the metachromic dye is insufficient to produce a metachromic shift. The addition of the enzyme and any required salt does not interfere with the measurement of glycosaminoglycan content because the glycosaminoglycan analyzer device of the present invention removes these interfering substances. Identification of the type of GAG present in the sample can, for example, be used to diagnose the type of MPS disorder the patient is suffering from or can be used to monitor more accurately the GAG of interest.

Separation Apparatus and Methods

The separation apparatus or cartridge removes substances that interfere with accurate measurements of glycosaminoglycan content (interfering substances), such as cells, proteins, nucleic acids, small contaminating molecules, and salt, preferably in as few steps as possible. The advantage of removing such interfering substances is that the accuracy of glycosaminoglycan measurement is greatly improved. Salt, such as sodium chloride, introduces a positive error and proteins, including but not limited to albumin, introduce negative error. Other compounds known to introduce error include sodium dodecyl sulfate (SDS), methanol, acetonitrile and BaOH. Salts and proteins are known to comprise a variable portion of a normal urine sample and an even larger variability exists in pathologic urine samples.

In one preferred embodiment, the separation apparatus comprises one or more ion exchange resins that separate glycosaminoglycans from interfering substances using a one-step wash. The diluted sample (body fluid sample mixed with buffered isotonic solution (“sample loading solution”, e.g., 200 mM NaCl, pH 8.3)) is infused through an ion-exchange cartridge under conditions that allow the glycosaminoglycans present in the sample to adsorb to the ion exchange media. Because cells and most interfering substances do not adsorb to the media, they are thereby separated from the glycosaminoglycans and discarded. Under these conditions, some interfering proteins may adsorb to the media; such residual protein is removed and discarded with a wash of buffered intermediate-concentration salt solution (“column wash solution”, e.g., 400 mM NaCl, pH 8.3). This intermediate salt washing step also removes nucleic acids, neutral compounds and other small contaminating compounds.

The separated glycosaminoglycans can then be mobilized from the cartridge toward the detection apparatus by washing with a buffered, high-concentration salt solution (“eluent”, e.g., 1000 mM NaCl, pH 8.3).

One of ordinary skill in the art can select ion exchange resin(s) suitable for use in the device of the invention. Desirable properties of such resins include mechanical stability, e.g., a resin that is not compressible and that does not shrink or swell in response to different solvents, ability to be highly substituted with cationic groups, and desirable flow characteristics. For example, resin properties may be chosen based on particle size to control system backpressure or to increase sample interaction as the sample flows through. Desirable flow characterisctics can also depend on the potential size of particles that may flow through the cartridge, e.g. in applications where the sample is blood a larger particle resin will allow flow of blood cells through a bed of resin particles better than a small resin particle bed which may clog with cells, whereas where the sample is urine no particles would be expected in a centrifuged sample so a small resin particle could be used.]. Suitable ion exchange resins include methacrylate resins such as TosoHaas (catalog number 43205), Toyopearl Super Q-650M, 56 micron anion-exchange resin; Sephadex; crosslinked cellulose; or silica. Suitable cationic groups for substitution may include quaternary amines, or primary amines at low pH. In addition to ion exchange chromotography, glycosaminoglycans may be separated from interfering proteins using techniques including, but not limited to, precipitation of proteins using, e.g., ethanol, trichloroacetic acid, or acetonitrile; reversed phase HPLC or solid phase extraction; size exclusion chromatography (which is based on the tendency of linear glycosaminoglycans to elute more quickly than globular proteins); cation exchange chromatography by HPLC or solid phase extraction; or precipitation of glycosaminoglycans using, for example, cetylpyridinium chloride or alcian blue, leaving the protein in solution. Precipitation methods may require centrifugation and/or phase separation steps.

Ion-exchange chromatography relies on the affinity of a substance for the exchanger, the affinity depending on both the electrical properties of the material and the relative affinity of other charged substances in the solvent. Hence, bound material can be eluted by changing the pH, thus altering the charge of the material, or by adding competing materials, salts being an example. The principle of ion-exchange chromatography is that charged molecules adsorb to ion exchangers reversibly so that molecules can be bound or eluted by changing the ionic environment. Separation using ion exchangers is usually accomplished in two stages: first, the substances to be separated (e.g., glycosaminoglycans) are bound to the exchanger, using conditions that give stable and tight binding; then the exchanger column is eluted with buffers of different pH, ionic strength, or composition, and the components of the buffer compete with the bound material for the binding sites.

An ion exchanger is usually a three-dimensional network or matrix that contains covalently-linked charge groups. If a group is negatively charged, it will exchange positive ions and is a cation exchanger. A typical group used in cation exchangers is the sulfonic group, SO³⁻. If an H⁺ is bound to the group, the exchanger is said to be in the acid form; it can, for example, exchange on H⁺ for one Na⁺ or two H⁺ for one Ca²⁺. The sulfonic acid group is a strongly acidic cation exchanger. Other commonly used groups are phenolic hydroxyl and carboxyl, both weakly acidic cation exchangers. If the charged group is positive—for example, a quaternary amino group—it is a strongly basic anion exchanger. The most common weakly basic anion exchangers are aromatic or aliphatic amino groups.

The matrix can be made of various materials. Commonly used materials are dextran, cellulose, agarose, and copolymers of styrene and vinylbenzene in which the divinylbenzene both cross-links the polystyrene strands and contains the charged groups. Table 1 gives the composition of many ion exchangers.

The total capacity of an ion exchanger measures its ability to take up exchangeable groups per milligram of dry weight. This number is supplied by the manufacturer and is important because, if the capacity is exceeded, ions will pass through the column without binding. Exemplary ion exchangers with their functional groups are identified in the following Table.

TABLE 1
Commercially Available Ion Exchange Resins

| Matrix | Exchanger | Functional Group | Tradename |
|---|---|---|---|
| Dextran | Strong Cationic | Sulfopropyl | SP-Sephadex |
| | Weak Cationic | Carboxymethyl | CM-Sephadex |
| | Strong Anionic | Diethyl-(2- | QAE-Sephadex |
| | | hydroxypropyl)- | |
| | | aminoethyl | |
| | Weak Anionic | Diethylaminoethyl | DEAE- |
| | | | Sephadex |
| Cellulose | Cationic | Carboxymethyl | CM-Cellulose |
| | Cationic | Phospho | P-cel |
| | Anionic | Diethylaminoethyl | DEAE- |
| | | | cellulose |
| | Anionic | Polyethylenimine | PEI-Cellulose |
| | Anionic | Benzoylated- | DEAE(BND)- |
| | | naphthoylated, | cellulose |
| | | deiethylaminoethyl | |
| | Anionic | p-Aminobenzyl | PAB-cellulose |
| Styrene- | Strong Cationic | Sulfonic acid | AG 50 |
| divinyl- | | | |
| benzene | | | |
| | Strong Anionic | | AG 1-Source |
| | | | 15Q |
| | Strong Cationic + | Sulfonic acid + | AG 501 |
| | Strong Anionic | Tetramethyl- | |
| | | ammonium | |
| Acrylic | Weak Cationic | Carboxylic | Bio-Rex 70 |
| | Strong Anionic | Trimethyl- | E. Merk |
| | | aminoethyl | |
| | Strong Anionic | Trimethylamino | Toso Haas |
| | | group | TSK-Gel- |
| | | | Q-5PW |
| Phenolic | Strong Cationic | Sulfonic acid | Bio-Rex 40 |
| Expoxyamine | Weak Anionic | Tertiary amino | AG-3 |

The porosity of the matrix is an important feature because the charged groups are both inside and outside the matrix and because the matrix also acts as a molecular sieve. Large molecules may be unable to penetrate the pores, so the capacity will decease with increasing molecular dimensions. The porosity of the polystyrene-based resins is determined by the amount of cross-linking by the divinylbenzene (porosity decreases with increasing amounts of divinylbenzene). With the Dowex and AG series, the percentage of divinylbenzene is indicated by a number after an X-hence, Dowex 50-X8 is 8% divinylbenzene.

Ion exchangers come in a variety of particle sizes, called mesh size. Finer mesh ion exchange resins have an increased surface-to-volume ratio, and therefore, increased capacity and decreased time for exchange to occur for a given volume of the exchanger. On the other hand, fine mesh produces a slow flow rate, which can increase diffusional spreading.

There are a number of choices to be made when employing ion exchange chromatography as a technique. The first choice to be made is whether the exchanger is to be anionic or cationic. If the materials to be bound to the column have a single charge (i.e., either plus or minus), the choice is clear. However, many substances (e.g., proteins, viruses), carry both negative and positive charges and the net charge depends on the pH. In such cases, the primary factor is the stability of the substance at various pH values. Most proteins have a pH range of stability (i.e., in which they do not denature) in which they are either positively or negatively charged. Hence, if a protein is stable at pH values above the isoelectric point, an anion exchanger should be used; if stable at values below the isoelectric point, a cation exchanger is required.

The choice between strong and weak exchangers is also based on the effect of pH on charge and stability. For example, if a weakly ionized substance that requires very low or high pH for ionization is chromatographed, a strong ion exchanger is called for because it functions over the entire pH range. However, if the substance is labile, weak ion exchangers are preferable because strong exchangers are often capable of distorting a molecule so much that the molecule denatures. The pH at which the substance is stable must, of course, be matched to the narrow range of pH in which a particular weak exchanger is charged. Weak ion exchangers are also excellent for the separation of molecules with a high charge from those with a small charge, because the weakly charged ions usually fail to bind. Weak exchangers also show greater resolution of substances if charge differences are very small. If a macromolecule has a very strong charge, it may be impossible to elute from a strong exchanger and a weak exchanger again may be preferable. In general, weak exchangers are more useful than strong exchangers.

The Sephadex and Bio-gel exchangers offer a particular advantage for macromolecules that are unstable in low ionic strength. Because the cross-linking in the support matrix of these materials maintains the insolubility of the matrix even if the matrix is highly polar, the density of ionizable groups can be made several times greater than is possible with cellulose ion exchangers. The increased charge density introduces an increased affinity so that adsorption can be carried out at higher ionic strengths. On the other hand, these exchangers retain some of their molecular sieving properties so that sometimes molecular weight differences annul the distribution caused by the charge differences; the molecular sieving effect may also enhance the separation.

The cellulose ion exchangers have proved to be the most effective for purifying large molecules such as proteins and polynucleotides. This is because the matrix is fibrous, and hence all functional groups are on the surface and available to even the largest molecules. In many cases, however, beaded forms such as DEAE-Sephacel and DEAE-Biogel P are more useful because there is a better flow rate and the molecular sieving effect aids in separation.

Buffers themselves consist of ions, and therefore, they can also exchange, and the pH equilibrium can be affected. To avoid these problems, generally cationic buffers a reused with anion exchangers and anionic buffers with cation exchangers. Because ionic strength is a factor in binding, a buffer should be chosen that has a high buffering capacity so that its ionic strength need not be too high.

Detection Apparatus and Methods

The glycosaminoglycans that have been separated from the interfering substances are combined with a detection reagent that binds to the glycosaminoglycans. The relative amount of glycosaminoglycans is then determined by detecting the amount of binding that occurs, compared to values detected for standards having a known concentration of glycosaminoglycans. Two, three, four or more standards may be used; typically values from three different standards are used to produce a standard curve against which the value for the sample is compared. The detection apparatus used to detect the amount of binding will vary depending on the detection method.

In a preferred embodiment of the invention, detection of glycosaminoglycan content is carried out using dyes that bind to glycosaminoglycans to form dye-glycosaminoglycan complexes detectable by measuring absorption at certain wavelengths. Specifically, a measured volume of dye may be mixed with the separated glycosaminoglycans. The dye is preferably a metachromic dye that preferentially binds to the glycosaminoglycans. The ability of glycosaminoglycans to bind cationic dyes such as dimethylmethylene blue (DMMB) is due to the high level of sulfation of the glycosaminoglycans. The cationic dye binds to the anionic sulfate groups which causes a shift in the absorption spectrum of the dye, possibly due to complex chemical interactions that affect conformation of the dye molecules and interactions among neighboring dye molecules. Metachromic dyes are particularly preferred as detection agents because only the glycosaminoglycan-complexed dye undergoes the spectral shift; consequently there is no need to separate the uncomplexed dye from the complexed dye. The amount of glycosaminoglycan bound to the dye may be measured using colorimetric determination, such as spectrophotometry. Thus one preferred detection apparatus is a spectrophotometer.

Suitable dyes include any cationic dyes that bind to anionic polymers and, when bound, exhibit a shifted absorption spectrum. Exemplary dyes intended for use according to the invention, generally metachromic dyes, include, but are not limited to alcian blue, azure A, azure B, methylene blue, fuchsin, acridine orange, proflavine, neutral red, and brilliant cresyl blue. One particularly preferred dye is 1,9-dimethylene blue.

Other detection methods include combining the glycosaminoglycans with a labeled detection reagent that binds to the glycosaminoglycans. Preferably the detection reagent specifically binds to the glycosaminoglycans or binds primarily to glycosaminoglycans compared to other proteins. Suitable detection reagents are known in the art and include polycations such as poly-L-lysine or (Lys-Ala)n and antibodies that bind to glycosaminoglycans. See, e.g., U.S. Pat. No. 6,630,295 disclosing fluorescently labeled polycations and U.S. Pat. No. 6,228,598 describing antibodies to heparin sulfate glycosaminoglycans.

Detectable labels are known in the art and include fluorescent labels, luminescent labels, radioactive isotopes, positron emitting metals, paramagnetic metal ions and enzymes. Exemplary fluorophores include 8-aminonaphthalene, 1,3,6-trisulphonic acid, 1-amino-4-naphthalene sulfonic acid, 1-amino-6,8-disulphonic acid, 2-aminoacridone and lucifer yellow, umbelliferone, fluorescein, fluorescein isothiocyanate, rhodamine, dichlorotriazinylamine fluorescein, dansyl chloride or phycoerythrin. Examples of a luminescent label include luminal, luciferase, luciferin, and aequorin. One of ordinary skill in the art would be readily able to choose a suitable detection apparatus depending on the label to be detected, e.g. a fluorimeter, positron emission tomography, scintillation counter, etc.

Those skilled in the art may routinely obtain and substitute other detection reagents, dyes, fluorophores and other labels all within the scope of the present invention.

To minimize errors and obtain precise and accurate measurements of urinary glycosaminoglycans, samples are assayed in a matrix that is equivalent to that of the standards used to construct a standard curve. The analytes and standards are preferably compared under the same or similar conditions of pH, salt concentration, temperature and/or time. The most accurate results are obtained when all of these conditions are kept equivalent and constant. For example, because glycosaminoglycan-dye complexes aggregate and eventually precipitate over time, absorbances will decrease over time. Preferably absorbance is measured during the first minute after contacting the glycosaminoglycans with the dye. Temperature fluctuations were also observed to introduce error into the measurements, especially at low glycosaminoglycan concentrations. The absorbance of the GAG/dye complex solution increases with increasing temperature. Thus, preferably the device of the present invention includes a temperature control component for stabilizing the temperature of the sample analytes and standards prior to and during detection of glycoaminoglycan-dye complexes. Detection temperature could be varied as convenient; for example, temperatures between 20 and 37° C. would be suitable. In addition, periodic cleaning of the components that handle liquid inputs improves system performance by reducing levels of contaminants that increase baseline measurements.

In one embodiment of the device the temperature control component is a thermofoil heater, and the temperature in the measurement cell is set to a value above ambient temperature (34° C.) so that the measurement cell temperature can be regulated without incorporating facilities for both heating and cooling. Following dye-eluent mixing the solution is held in the measurement cell until the solution temperature reaches the set point; this equilibration may take 10 to 45 seconds depending on ambient temperatures and the temperature of the dye solution. This embodiment also incorporates an ethanol wash in each analysis cycle.

Different glycosaminoglycan types have different optimal pH and dye concentrations with respect to detection. For example, heparin detection may be optimal at a pH of 2.0, whereas detection of dermatan may be optimal at a pH of 2.5. Typically the pH is adjusted inversely to the degree of sulphation of the GAG. Dye concentration can be adjusted to suit the predominant range of GAG concentrations being analyzed, e.g. reduced dye concentration may be desirable for lower GAG concentrations. For the highest sulphated GAGs (e.g. Heparin) dye binding works well at lower pH. It was determined that dermatan required a higher pH and that the high pH also works well with heparin. Detection at lower pH thus will improve the specificity for heparin when using the device as a heparin monitor.

Measurement of the absorbance of the dye-glycosaminoglycan complex is carried out at an appropriate wavelength determinable by one of ordinary skill in the art, for example 592 mm. Measurement of absorbance at two or more wavelengths, for example, at 526+/−5 nm and 592+/−5 nm for DMMB, allows for detection of errors in the device function. Absorbance of glycosaminoglycan-DMMB complexes at 592 nm is inversely proportional to glycosaminoglycan concentration, while absorbance of glycosaminoglycan-DMMB complexes at 526 nm is directly proportional to glycosaminoglycan concentration. In other words, when a glycosaminoglycan is present, absorbance at 526 nm increases and absorbance at 592 nm decreases. Failure to observe changes at both of these wavelengths indicates a false positive error in the system. These two nominal wavelengths are both readily available and economical LED light sources.

In one embodiment, absorbance is calculated from raw detector output as follows:
sample.Abs_{—}w_{—}Dark=Abs(Log((sample.Mix−sample.Dark)/((sample.Clear*2.878788)−sample.Dark))/Log(10))/0.726, where
sample.Mix=raw detector data for mix of dye/glycosaminoglycan
sample.Dark=raw detector data for electronic background signal
sample.Clear=raw detector data for reference (solution blank)

2.878788 is a correction factor for integration time between sample.Clear and sample.Mix that may vary depending on the construction of the device.

0.726 is a correction factor to correct detector pathlength to 1 cm, and also may vary depending on the size of the detection chamber.

At least three standards of known glycosaminoglycan concentration are assayed to create a standard curve. Four or more standards may also be used. For example, standards of 50, 150 and 450 mg/mL dermatan sulfate may be used. As another example, standards of 100, 200 and 400 mg/mL may be used. Conventional regression calculations are used to create the standard curve for calibration.

Referring again to FIG. 1, the eluted glycosaminoglycans accumulate in a syringe 102 after passing out of the cartridge 136 and through the solenoid valve 138. The eluate may not be homogeneous and, therefore, may need to be mixed so that any sample portion is of the same glycosaminoglycan concentration. A chamber or port 122 may be provided for introducing a diluent into the substantially separated glycosaminoglycan sample. A portion of the eluate is then mixed with the DMMB dye solution. A chamber or mixing line 134 for mixing the glycosaminoglycans or a portion of the eluate with diluent and/or a suitable dye may provided, which may pass the eluate between the first and second syringe pumps 102, 104. A lamp 142 and detector 144 are located in immediate proximity to the first syringe pump 102 for detecting glycoaminoglycans molecules bound to a detectable dye. The first syringe pump 102 may be glass, the plunger drawn out of the spectrometer path during reading of the sample. Fiber optics may be used to conduct light from the light source to the syringe body, and the transmitted light may be received by another fiber optic and sent to the spectrometer. A waste port 124 may be provided for removing waste materials including unbound dye from the glycosaminoglycan sample.

The components of the system 100 are known and available. For example, syringe pumps 102, 104 can be Kloehn, Ltd. model 50300, with optional expansion, part no. 17737. The first rotary valve 106 can be a Kloehn Ltd. part no. P0107, while the second rotary valve 108 can be a Kloehn Ltd. part no. 17620.

Glycosaminoglycan concentration may be reported in units/mL or micrograms/mL on a screen and/or a printed tape. Data may also be recorded to a floppy disk accessible through the rear panel of the instrument. In addition to the functions described above, the glycosaminoglycan autoanalyzer may rinse all fluidic paths, and regenerate the glycosaminoglycan capture cartridge. Rinsing liquid handling components with 70% ethanol improves system performance by reducing baseline increasing contaminant levels, and is recommended. Ethanol 118 may be drawn into syringe pump one 102 through port 114a. Ethanol may then reach syringe pump two 104 via a mixing line 134, or via port 114g by way of the solenoid valve 138, the glycosaminoglycan capture cartridge 136, and port 116c.

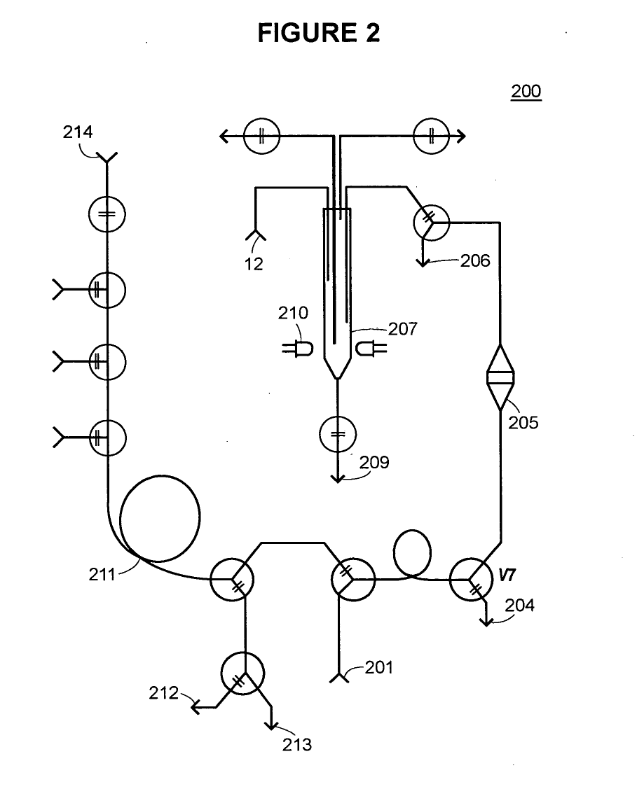

Turning to FIG. 2, a schematic representation 200 of the elements of a second automated glycosaminoglycan analyzer device according to the present invention, is provided. Specifically, a sample port 201 is provided for introducing a body fluid sample. A port 204 is also provided for waste removal. A glycosaminoglycan capture cartridge 205 is provided for substantially separating glycosaminoglycans from the remaining components in the body fluid. A second port 206 is provided for removing waste materials including the remaining components of the body fluid. A chamber or colorimetric processor 207 for mixing the glycosaminoglycans with a suitable dye is also provided. Optionally, a port 209 is provided for removing waste materials, including unbound dye from the glycosaminoglycan-dye sample. A detector 210 is provided for detecting glycosaminoglycan molecules bound to detectable dye. Additionally, means 211 are provided for measuring reagents for mixing with body fluid samples and/or with substantially separated glycosaminoglycan samples, including a reagent measuring loop. Reagent may accumulate in the reagent measuring loop 211 until the loop overflows through an associated waste port, for example waste port 213. In addition, glycosaminoglycan containing eluate may be measured using various length tubes or lines arranged to control the final volume of eluate transported to the mixer 207. Further, gas may be delivered via valve 220 for mixing the DMMB dye solution with the eluate. Glycosaminoglycan concentration can be measured by measuring the DMMB/glycosaminoglycan absorbance across the diameter of the mixing chamber 207. Means 212 may be provided for transporting such reagents to the body fluid sample or the substantially separated glycosaminoglycan sample, including transport lines. In addition, one or more waste ports 213 may be provided for removing excess reagents. Further, one or more ports 214 may be provided for introducing gas into the transport lines for priming the system after each sample run.

Those of skill in the art can provide transport means between ports, containers, chambers, cartridges, and detectors of the present invention using standard materials known to those skilled in the art. Likewise, syringes or other sampling means and any requisite valves for separating components may be made from standard materials known to those skilled in the art.

Those of skill in the art readily appreciate that the physical appearance and dimensions of devices within the scope of the present invention may be adapted as desired or preferred. Exemplary embodiments may include a front panel. The top half of the front panel is preferably a touch screen display. All operator input is entered using the touch screen display. The operator's finger may be used like a computer mouse to direct control of the device and to enter data. A single touch on the screen functions as a double click on a mouse. A fingertip dragged across the screen is the same as a click-and-drag mouse event. The lower portion of the front panel features the printer tape output slot and the sample holder.

Further, an exemplary device may include a right side panel. The lower right side panel is preferably a fluidic compartment access door. In one embodiment, the door may be opened by turning two wing nuts, for example, one-quarter turn counter-clockwise, releasing the door. The door may be hinged at the rear. A left side panel may also be provided that may include a reagent compartment access door. In one embodiment, the door may be opened by turning two wing nuts, for example, one-quarter turn counter-clockwise, releasing the door. The door may be hinged at the rear.

A rear panel may also be included. A power input module may be located on the upper left (viewing the instrument from the rear) corner. The power input module incorporates the fuse, power switch and receptacle for AC line voltage. A fan grill may be located in the upper center of the rear panel. A connector for a network cable may be located below the fan. DIN receptacles for an optional PS2 mouse and keyboard may be located to the left of the network cable. A 1.44 Mbyte floppy disk drive may be located below the mouse and keyboard drive.

Preferably, the device is designed for use on a countertop or cart top. Exemplary dimensions are about 14″ wide and about 17″ front to back and about 24″ above the countertop. A preferred power supply is 100 VAC to 230 VAC₁₋₆₀ cycle. An uninterruptible power supply is recommended.

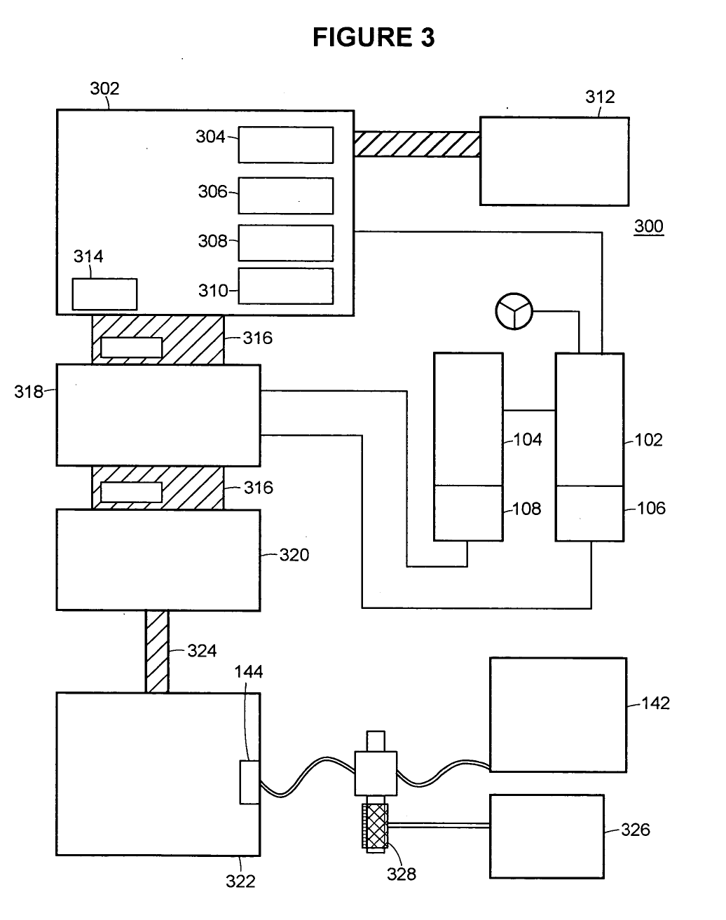

In another aspect, the invention provides a processor for operating the device. Referring to FIG. 3, a controller 300 for the system 100 is discussed and described. A PC 302 has a processor 304 for executing commands and operating on data stored in memory 306. The memory 306 may include both non-volatile memory 308, such as read only memory (ROM) and electrically erasable programmable read only memory (EEPROM) and volatile memory 310 such as random access memory (RAM) in one or more of several common forms. Removable storage 312, for example, a floppy disk drive, can be used for both executable code and data. The PC 302 also includes a display 314. In one embodiment, the display 314 also includes a touch screen for capturing input from an operator.

As further illustrated in FIG. 3, the PC 302 has a standard ISA bus 316 for supporting ISA plug-in cards. A digital I/O card 318 for command and control of the syringe pumps 102, 104 and the rotary valves 106, 108 is coupled to one ISA slot. An analog to digital (A/D) converter 320 is similarly plugged into another ISA slot. The ISA bus 316 supports high speed data transfers between the processor 304 and I/O and A/D cards 318, 320.

A spectrometer 322 is coupled to the A/D card 320 by a cable 324. The lamp 142 is arranged for passing light through the sample to the detector 144 associated with the spectrometer 322. A temperature controller 326 may act independently or under control of the PC to adjust a heater 328 to regulate the sample under test at an appropriate temperature, for example, 34 degrees Celsius.

The programming of the PC 302 for is within the capabilities of one of ordinary skill in software development, given the desired operating procedure described above. The ‘C’ language provides a suitable development environment for such a program, although critical control routines may be done in assembler as necessary.

The components of the controller 300 are known and available.

The analyzer according to the present invention is preferably an automated analyzer for assaying for the presence of glycosaminoglycans, for analyzing medical samples, or human or animal-derived material for the purposes of medical diagnosis, for monitoring the synthesis of glycosaminoglycans, and for monitoring a system that uses or uptakes glycosaminoglycans. An analyzer according to the present invention may be used in conjunction with kits that include standards, buffers, glycosaminoglycan detection dyes, and instructions for carrying out the methods of the present invention. An analyzer according to the present invention is especially useful in monitoring patients undergoing heparin therapy such as during surgical procedures. Moreover, an analyzer according to the present invention may be used to measure or monitor endogenous glycosaminoglycan levels and is therefore useful in diagnosing or screening for risk of osteoporosis and/or kidney disease.

The present invention provides testing and/or analysis of glycosaminoglycans, including heparin and heparin sulfate, to determine the presence and quantity of these substances in body fluids such as blood. The present invention provides methods for detecting and measuring urinary glycosaminoglycans and provides for establishing a predictable relationship between urinary heparin levels and plasma heparin levels.

Still further, a glycosaminoglycan automated analyzer and methods according to the present invention may be used to measure specific species of glycosaminoglycan. Specifically, a bodily fluid sample may be divided, one portion of which is pretreated with an enzyme that degrades a certain glycosaminoglycan species. The total glycosaminoglycan content of the treated and untreated samples may then be determined. Comparison of the glycosaminoglycan content detected in pretreated and untreated samples will reveal the amount of glycosaminoglycan degraded by the species specific enzyme, in turn indicating the amount of the specific species of glycosaminoglycan degraded. In this manner, measurement of certain glycosaminoglycans may give confirming evidence that a patient bears the biochemical marker for a particular disease.

### EXAMPLE 1

### Systems, Materials, and Methods

An exemplary device (referenced as “GAGbot”) was constructed that quantifies the total sulfated glycosaminoglycans in urine, normalized with the concentration of creatinine in urine. The device relies on the spectrophotometric detection (at 592 nm) of metachromatic changes in 1,9-dimethylmethylene-blue (DMMB) that occur after the formation of the GAG-DMMB complexes. During the automated assay process, urinary GAGs are separated from interfering substances via an ion-exchange column and eluting with different salt concentrations. The column cartridge is packed with Toyopearl™ Super Q-650M resin. The device is calibrated with dermatan sulfate standards at 100, 200 and 400 mg/mL. Urine samples or controls are manually loaded on the machine in volumes of 100 mL, 200 mL, 400 mL or 1200 mL. The sample is taken up by the analyzer, diluted with 200 NaCl, 20 mM Tris, pH 8.3 and loaded onto the ion exchange column. The column is washed with 400 mM NaCl, 20 mM Tris, pH 8.3. The glycosaminoglycans are eluted from the column by 1200 mM NaCl, 20 mM Tris, pH 8.3, mixed with acidified DMMB, and detected by the absorbance of 592 nm light. Due to the low pH and prior chromatographic separation, only GAGs from the urine are able to interact with the DMMB. From the raw absorbance data and sample loading volume, the instrument software calculates mg/mL concentration values for total GAGs using the standard curve of dermatan standards.

In the GAGbot device, the cycle of operation is as follows.

Introduction of sample—A sample (urine) of 0.05 mL, 0.2 mL, or 1.5 mL is put into the sample holder. GAGbot dilutes the sample to 2.0 mL with a buffered salt solution (“Diluent”).

Extraction of glycosaminoglycan—Diluted urine is infused through a proprietary ion-exchange cartridge in order to extract glycosaminoglycan from the sample solution. Under the conditions used, some interfering proteins adsorb to the media in the ion-exchange cartridge. Most interfering substances are separated from the glycosaminoglycan and sent to waste.

Removal of interfering proteins—Residual protein in the glycosaminoglycan capture cartridge is removed with a wash of buffered intermediate-concentration salt solution (“Buffer”).

Elution of glycosaminoglycan from the glycosaminoglycan capture cartridge—glycosaminoglycan is mobilized toward the colorimetry processor with a buffered, high-concentration salt solution (“Eluent”).

Dye Mixing—Eluted glycosaminoglycan is well mixed and a measured portion of the eluted glycosaminoglycan is saved for the colorimetric determination. A measured volume of dye (“Indicator”) is mixed with the eluted glycosaminoglycan and mixed. The dye-glycosaminoglycan complex is formed instantaneously and the transmittance of 595 nm light, through the solution of mixed dye and glycosaminoglycan, is measured.

Cleanup—GAGbot sends the dye solution to waste, rinses all fluidic circuits, and regenerates the glycosaminoglycan capture cartridge.

Data Output—glycosaminoglycan concentration is reported in uG/mL on both the screen and on printed tape. Data is also recorded to a floppy disk accessible via the rear panel of the instrument.

Performance

Specificity—The device has been demonstrated to specifically measure polymeric sulfated glycosaminoglycans. Hyaluronic acid and chondroitin disaccharides do not produce a detectable response. The device is most sensitive to heparin.

Chondroitin sulfates A and C, keratan sulphate, and dermatan sulfate produce responses of a nearly equal level. This makes GAGbot useful in screening a large variety of MPS disorders where glycosaminoglycan levels are expected to be elevated in urine. GAGbot's advantage over enzymatic tests for specific enzyme deficiencies is obvious since GAGbot can, in a single test, reveal abnormally high glycosaminoglycan levels.

Sensitivity—The device has a measurement range of 6.7 ug/mL to 1800 ug/mL.

Throughput—Minutes/cycle is 6 minutes 40 seconds/sample. The cycle time of 6:40 minutes is not a complete indication of throughput. To start the machine requires 30 minutes of warm-up time for electronics and at least one run of a 30 minute “start-up” routine followed by a 18 minute calibration routine. Combined with end-of-day rinsing and shutdown procedures 2 hours of each working day should be allotted to machine calibration and maintenance.

Reagents

One embodiment of a device according to the present invention uses and four reagents: a Diluent, a Buffer, an Eluent, and an Indicator (prepared by mixing a dye solution with a dye diluent). Exemplary formulations are set forth below. Similar or substantially equivalent formulations with other agents such as salts or dyes may be easily and routinely prepared by those of skill in the art. For example, as an alternative to using two separate Buffer and Eluent salt solutions, a single high salt solution could be diluted with the appropriate amount of Diluent to produce a solution of the desired salt concentration.

Diluent (20 mM Tris Base)
Components Formula Quantity (in 1000 mL)
Trizma Base 2.422 g
Sodium Azide 0.25 g
Adjust with dilute (e.g. 1N) HCl to pH 8.3.

Buffer (400 mM NaCl, 20 mM Tris Base)
Components Formula Quantity (in 1000 mL)
Sodium Chloride 23.4 g
Trizma Base 2.422 g
Sodium Azide 0.25 g
Adjust with dilute (e.g. 1N) HCl to pH of 8.3.

Eluent (1000 mM NaCl, 20 mM Tris Base)
Components Formula Quantity (in 1000 mL)
Sodium Chloride 58.4 g
Trizma Base 2.422 g
Sodium Azide 0.25 g
Adjust with dilute (e.g. 1N) HCl to pH 8.3.

DMMB Dye Solution
Components Formula Quantity (in 1000 mL)
Absolute Ethanol 5.0 mL
Dimethylmethylene Blue Dye Powder 21.0 mg
Sodium Formate 2.0 g
Formic Acid Approx. 20 mL

The dye is prepared by stirring 21.0 milligrams of dimethylmethylene (DMMB) blue dye powder in 5.0 milliliters of absolute ethanol for at least one hour; this is added to 800 mL of DI water, then sodium formate is added. Adjust pH to 2.50 by titrating with Formic Acid.

DMMB Dye Solution Diluent
Components Formula Quantity (in 1000 mL)
Sodium Formate 2.0 g
Formic Acid Approx. 20 mL

The solution is prepared by dissolving sodium formate in water, adjusting pH to 2.50 by titrating with formic acid, and bringing to a final volume of IL with DI wter.

DMMB Solution (Indicator)

DMMB Solution is prepared by titrating DMMB Dye Solution with DMMB Solution Diluent and adjusting the optical density of the DMMB Dye Solution. Preparation of the “Indicator” solution may occur independent of the analyzer. Set the spectrophotometer wavelength to 592 nm and use DMMB Dye Solution Diluent to set the spectrophotometer zero reference.

Measure the optical density of the DMMB Dye Solution. If the A592 is 2.00+/−0.005 the DMMB Dye Solution requires no further adjustment. If the A592 is less than 1.95 discard the DMMB Dye Solution and reformulate the solution. If the A592 is greater than 2.05, adjust the A592 to 2.00 by titrating with DMMB Solution Diluent.

The operating software uses three screens: the introduction screen, the utilities screen and the run screen. The introduction screen is presented for approximately 5 seconds a startup and displays the version number and date. The utilities screen is the user interface for pre and post sample processing functions. The run screen is the interface for sample processing.

Utilities Screen Functions

Buttons:

Title Bar—the title bar displays the current version date of the controller software and the patient's name

Initialize—The initialize button sends the syringes and valves to a default position that any of the other functions of the run or utilities screens must begin from. Pressing the Initialize button is not usually necessary since initialization is built into the other functions. Initialize may be useful for troubleshooting and recovery from error events.

Enter Patient—Pressing the Enter Patient key will invoke the patient name entry keyboard. Enter the letters and numbers. Spaces can be inserted with the “_” key and the “BackSpace” key can be used to correct errors. Press the Enter key to save the patient name. The Cancel key allows escape from the keyboard without saving data.

Prime—Press the Prime key after any one reagent on GAGbot has been changed. GAGbot will purge air out of reagent supply lines by running a priming routine that purges all the supply lines. Remember that priming consumes approximately one sample run's volume of solutions.

Start Routine—The Start Routine runs five consecutive glycosaminoglycan analysis cycles. Use this function to prepare the GAGbot for calibration. The Start Routine will ensure that the cartridge is equilibrated and that the GAGbot will be at a stable operating temperature. The Start Routine consumes five cycles worth of reagents and takes approximately 30 minutes.

Run Standards—Pressing the Run Standards button initiates a process that performs measurements of 3 samples with known glycosaminoglycan concentrations. The data from these measurements are used in an algorithm that interprets the raw detector data into Unit per mL output. Accuracy of GAGbot's units/mL results depends on careful execution of running standards.

The three standards used are dermatan sulphate standards of 50, 150 and 450 mg/mL dermatan sulfate, or alternatively 100, 200 and 400 mg/mL dermatan sulphate. The standards are submitted to the GAGbot in 12 mm×75 mm disposable glass test tubes via the sample bay.

To calibrate, follow the above instructions for system start-up and perform a Prime and perform the Start Routine. Press the Run Standards button and follow the prompts. GAGbot will prompt for the three standard solutions. At the end of the Run Standards process the data for the calibration samples will be displayed along with the standard curve data and a pass/fail message. “Fail” is displayed if any one of the uG/mL values calculated using the equation for the standard curve is not within 15% of the nominal value(+/−20% for the lowest standard) for that standard. The absorbance values for each of the standards will also be displayed.

Enter Cal Values—The absorbance values provided by the Run Standards function can be entered manually using this function. Press the “Enter Cal Values” command button and follow the on-screen prompts.

Clean—The Clean function automates cleaning of the fluidics. The operator is instructed to fill the reagent bottles with water and to place a cleanser in a sample tube. After the cleanser has been used it is expelled and the operator is instructed by on-screen prompts to replace the sample tube with a water filled tube. This function requires approximately 30 minutes.

Reset Cycle Counter—The cycle counter is a box on the lower right of the screen. The number of runs performed since start-up or the last Cycle Counter reset is displayed in the cycle counter. Use the cycle counter to monitor waste production and reagent usage. A full set of reagents is enough for 30 analysis cycles. Press the “Reset Cycle Counter” button to return the cycle counter value to zero.

Initialize—“Initialize” sets the syringe pumps to their default home positions. Use this function for troubleshooting error conditions that might be caused by power problems or system malfunctions.

Shutdown—This function must be used before tuning of the power to GAGbot. The syringes will be parked in their home positions and the software will be closed in an orderly condition. During the shutdown procedure the operator will be prompted to choose between exiting to the operating system or exiting the software and powering down, the first choice is intended for service personnel only.

System Event Log—A log file of all operator actions is kept on the floppy disk and an identical log is kept on the controller's internal hard disk. Data for each sample analysis is also kept in these logs.

Instructions for Utilities Screen:

Log in Operator—Begin by identifying yourself as the operator. Press the Log in Operator key. This will be followed by a prompt to enter your name, via the on-screen keyboard. The entry is saved in a log file.

Prime—Press this key to fill reagent delivery lines when the GAGbot is started or when one or more reagents are refilled.

Start Routine—This key initiates the start routine that will prepare GAGbot for operation. The start routine performs five consecutive measurement cycles in order to equilibrate the ion-exchange cartridge and to bring the instrument to operating temperature.

Run Standards—Press this key to start the calibration process. The operator will be directed through the process of submitting three quantitative standard solutions (e.g., 50 mg/mL, 150 mg/mL and 450 mg/mL dermatan sulphate in water) for measurement and generation of a standard curve. Slope and intercept data derived from the standard curve are used for quantitation of subsequent samples. Execute the Prime and Start Routine functions before the Run Standard function. Record the onscreen results after the calibration is finished. If the calibration fails take remedial action and recalibrate.

Enter Cal Values—This key starts a series of prompts for reentry of calibration standards values. Use this function if GAGbot operation is interrupted by a short-term power outage or operating system malfunction. Evaluate whether a warm-up the Start Routine should be performed before using this function, if yes, the Run Standards function should be employed instead.

Clean—Pressing Clean starts a cleaning process. The operator is prompted to put a cleaning solution in the sample bay. At the end of the cleaning process the operator is prompted to place water in the sample bay (see maintenance for more information on cleaning).

Reset Cycle Counter—When this key is pressed the cycle counter (See Run Screen) is set to zero. Reset the cycle counter after replenishing reagents.

Initialize—Press Initialize to set GAGbot's syringe pumps to their home positions and set valves to default states. This is not a routine operation; initialize is useful for error recovery and troubleshooting.

Shut Down—Press the Shut Down key to set pumps and valves in their default positions before tuning off power to GAGbot. Shut Down invokes an orderly shut down of GAGbot by emptying the pumps and setting valves off, the operating system is shutdown and the will be prompted to turn off the power.

Daily, remove the reagents and replace the bottles with bottles of HPLC or Milli-Q grade water. Perform a prime and run a blank run to rinse all valves, syringes and tubing. Failure to do so will result in crystalization of salt in the valves and lead to leaks.

Run Screen—Pressing this key causes the Utilities Screen to be replaced by the Run Screen.

Results—Results are displayed in tabular form in the results field. The most current result is presented at the top of the table in bold type. As new results are posted, the previous results move downward in the table and change to standard type. For each result the sample name, concentration in micrograms per milliliter, and sample volume in microliters are displayed. For troubleshooting and diagnosis of malfunctions, the results table can be expanded to display addition raw data.

Run Sample—Pressed this key to initiate a glycosaminoglycan measurement process. The operator will be prompted to input the sample name and volume. If the GAGbot is not calibrated a warning will be presented.

Enter Comment—Comments can be entered into a log file (See software section) using an on-screen keyboard. All comments include the system date and time. The log file is recorded to the floppy disk and also to the system hard disk.

Utilities Screen—Pressing this key causes the Run Screen to be replaced by the Utilities Screen.

Status—This field displays a description of the current step in an analysis procedure. The status field keeps the operator advised of the progress of GAGbot during measurement procedures and other procedures.

Cycles—This field displays the number of analysis cycles performed since GAGbot was turned on or since the cycle counter was reset (See also “Reset Cycle Counter” on the Utilities screen.). The cycle counter aids the operator in monitoring reagent usage.

The following components are exemplary:

Pump 1: Kloehn, Ltd., Model 50300, 24000 step, Optional RS-232 adapter, Firmware version 1.17, Optional expansion Part number 17737 non-volatile memory of 7750 bytes.

Pump 2: Kloehn, Ltd., Model 50300, 24000 step, No optional RS-232 adapter, Firmware version 1.17, Optional expansion Part number 17737 non-volatile memory of 7750 bytes.

Rotary Valve, Pump 1: Kloehn Ltd., Part number P0107, 8-way rotary distribution valve, Ceramic, 0.059″ ports with ¼′-28 threads.

Rotary Valve, Pump 2: Kloehn Ltd., Part number 17620, 8-way rotary distribution valve, PTFE plug, Kel-F stator, 0.059″ ports with ¼′-28 threads.

Syringes, Pump 1 and Pump 2: Kloehn Ltd., Part number 18452, 2.5 mL Zero-dead-volume, polyethylene plunger.

Solenoid Valve: The Lee Company, Part number LFRX0500500BC, 3-way, nominal 0.060 ports, ¼″-28 threads, 12 VDC, PEEK and PTFE wetted surfaces.

Spectrometer: Ocean Optics Inc., Model S2000-TR, Temperature regulated fiber optic spectrometer, 1200 line holographic grating (VIS), 475-750 nm bandwidth, 10 um slit, with factory nonlinearity report.

### EXAMPLE 2

### Determination of Heparin Content in Plasma Samples

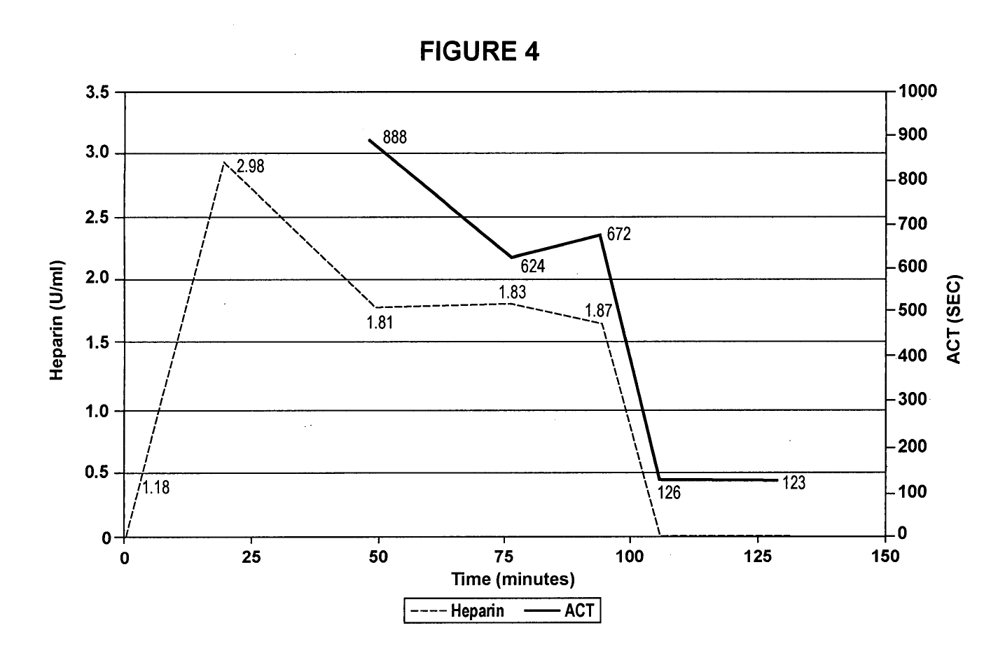

FIG. 4 was generated using data produced by an embodiment of the GAGbot configured specifically for measuring heparin in whole blood. The measurement range of the heparin analyzer included 0.5 to 6.0 units/mL (1 unit=160 micrograms) of heparin in whole blood.

The sample used was 1.0 mL anticoagulated whole blood; if the blood did not contain heparin it was anticoagulated using EDTA or citrate.

Calibration required a water blank, and heparin diluted in water to 0.5 units/mL and 6.0 units/mL. Calibration required the use of the same lot of heparin as was to be used for the monitored surgical procedure as it is expected that instrument response might be variable to different manufacturers and lots of heparin. Heparin manufacturers may specify differing anticoagulant activity for the same mass quantity of heparin so the capability of accomodating such variability has to be part of the instrument design.

Reagent Formulations: Buffer was aqueous 200 millimolar sodium chloride, 20 millimolar Trizma base titrated to pH 8.3 using dilute hydrochloric acid. Diluent was aqueous 20 millimolar Trizma base titrated to pH 8.3 using dilute hydrochloric acid. Eluent was aqueous 1200 millimolar sodium chloride, 20 millimolar Trizma base titrated to pH 8.3 using dilute hydrochloric acid.

Indicator was prepared by combining 21.3 milligrams dimethylmethylene blue dissolved in 5 milliliters ethanol, 2.025 grams sodium formate dissolved in 800 milliliters water. The solution was titrated to pH 2.00 with neat formic acid, q.s. to 1 liter. A595 (assuming 1 cm path)=1.9 approximately.

Isolation cartridge—A larger pore size frit material was used to permit passage of white blood cells and erythrocytes through the cartridge. This also required the use of a large particle size ion-exchange resin.
Resin: Toyopearl Super Q-650C, nominal particle size 100 um
Frit Material: Porex Expanded Polyethylene nominal pore size 45 um Aliquots of blood analyzed by the heparin monitor were also used for Activated Clotting Time (ACT) measurements, an indirect method of assesing anticoagulation. Data for ACTs were plotted against the heparin measurements in FIG. 4 and show good correlation.

### EXAMPLE 3

### Determination of Glycosaminoglycan Content in Urine Samples

Total urinary glycosaminoglycan excretion in samples of patients diagnosed with MPS I was measured by either manual Alcian blue method or using the device of the present invention. Briefly, the Alcian blue assay involves precipitation of glycosaminoglycans by Alcian blue in high salt (0.4M guanidine HCl), low pH (pH 1.75) in the presence of Triton X-100 (0.25%). The glycosaminoglycans are first isolated as precipitates of Alcian blue-glycosaminoglycan complexes, and then solubilized for spectrophotometric quantitation of absorbance at 600 nm. The higher the glycosaminoglycan content, the bluer the sample will be and the higher the absorbance. The absorbance of the Alcian blue-glycosaminoglycan complex is directly proportional to glycosaminoglycan concentration. This technique is highly sensitive and can detect concentrations of glycosaminoglycan ranging from 10 to 600 ug/ml, in a volume of 50 ul of urine, with accuracy. However, this precipitation technique is inconvenient, technically cumbersome, labor intensive and time consuming. The automated glycosaminoglycan analyzer device shortened the process from hours to only about 6 minutes. As described elsewhere herein, the glycosaminoglycans are first isolated from urine via an ion exchange chromatographic cartridge by changing salt concentration and then estimated spectrophotometrically at 592 nm based on the shift of the absorbance maxima of DMMB dye from 590-600 nm to 530 nm. The DMMB solution turns from blue to purple and pink immediately after addition of isolated glycosaminoglycans due to formation of a soluble metachromatic complex. The higher the glycosaminoglycan content, the pinker the sample will be, corresponding to a lower absorbance reading. The absorbance of the glycosaminoglycan-DMMB complex is inversely proportional to the glycosaminoglycan concentration. This method proved to be suitable for quantitation of urinary glycosaminoglycans between 8 and 800 ug/ml of urine. A graph plotting glycosaminoglycan measurements determined using the manual Alcian blue method vs. glycosaminoglycan measurements determined using the automated glycosaminoglycan analyzer is shown as FIG. 6. A correlation analysis (y=1.504x-1.5777, R²=0.9353) showed that normal glycosaminoglycan values measured by the two methods are positively correlated about 97% of the time.

### EXAMPLE 4

### Treatment of Samples Using Glycosaminoglycan-Specific Enzyme

Using the table below, enzymes are selected for the pretreatment of patient samples based on ability to deplete the target class glycosaminoglycan specifically, and the differential between digested and undigested sample glycosaminoglycan content is determined. As an example, for diagnosing MPS VI (Maroteaux-Lamy), the sample is treated with an enzyme (such as Chondroitinase B) that depolymerizes only dermatan sulfate into oligosaccharide units too small to be detected by a glycosaminoglycan analyzer device. A large differential between the measured glycosaminoglycan content of the sample before and after enzyme digestion confirms that the sample contains largely dermatan sulfate, the marker for MPS VI. The table below describes which urinary glycosaminoglycans are specific markers for a MPS.

TABLE 1
Enzyme Defects and Excretion Products of
Mucopolysaccharidoses

| | | Urinary |
|---|---|---|
| | | glycosaminoglycan |
| Disease | Enzyme deficiency | marker |
| MPS IH | α-L-Iduronidase | Dermatan sulfate, |
| (Hurler syndrome) | | heparan sulfate |
| MPS I-H/S | α-L-Iduronidase | Dermatan sulfate, |
| (Hurler-Scheie | | heparan sulfate |
| syndrome) | | |
| MPS IS | α-L-Iduronidase | Dermatan sulfate, |
| (Scheie | | heparan sulfate |
| syndrome) | | |
| MPS II-A | Iduronate sulfatase | Dermatan sulfate, |
| (Hunter syndrome, | | heparan sulfate |
| severe) | | |
| MPS II-B | Iduronate sulfatase | Dermatan sulfate, |
| (Hunter syndrome, mild) | | heparan sulfate |
| MPS III-A | Heparan N-sulfatase | Heparan sulfate |
| (Sanfilippo syndrome A) | | |
| MPS III-B | N-acetylglucosaminidase | Heparan sulfate |
| (Sanfilippo syndrome B) | | |
| MPS III-C | Acetyl-coenzyme A: α- | Heparan sulfate |
| (Sanfilippo syndrome C) | glucosamine-N- | |
| | acetyltransferase | |
| MPS III-D | N-acetyl α- | Heparan sulfate |
| (Sanfilippo syndrome D) | glucosamine-6- | |
| | sulfatase | |
| MPS IV-A | N-acetylgalactosamine-6- | Keratan sulfate |
| (Morquio syndrome A) | sulfatase | |
| MPS IV-B | B-galactosidase | Keratan sulfate |
| (Morquio syndrome B) | | |
| MPS VI | N-acetylgalactosamine-4- | Dermatan sulfate |
| (Maroteaux-Lamy) | sulfatase | |
| MPS VII | B-glucuronidase | Dermatan sulfate, |
| (Sly syndrome) | | heparan sulfate |

The following briefly describes enzymes suitable for digesting various glycosaminoglycans and illustrative protocols for digestion. Suitable conditions will be easily determined by those skilled in the art.

Heparinase degrades the most highly sulphated species, Heparitanase II will degrade heparin with relatively low sulphation and highly sulfated heparan while Heparitanase and Heparitanase 1 degrade the less sulphated heparan species (see http://www.seikagaku-hit.com/english/02tech/enz/04ko/p01.htm for a chart illustrating the substrate/enzyme continuum.

Keratanased (E.C 3.2.1.103)

The keratanase digestion of keratan sulfate is done in 0.2M sodium acetate buffer pH 7.2 and 5 mM 2,3 dehydro-2-deoxy-N-acetylneuraminic acid (a neuramimidase inhibitor). Enzyme is added at 1 unit per 2.3 mg of KS. The digestion is performed at 37 C for 24 hours. Following this digestion the enzyme is inactivated by boiling the sample for 2 minutes.

Keratanase II (Bacillus sp.)

Keratanase II, an endo-beta-N-acetylglucosaminidase, cleaves the beta(1-3)-glycosidic bond of a 6-O-sulfated N-acetyl-glucosamine in keratan sulfates. Tetra- and disaccharides are generated from the sulfated poly-N-acetyllactosamine repeat region, and in the case of the N-acetyl-neuraminic acid-containing capping oligosaccharides, pentasaccharides are recovered. Keratan sulfate is digested at 1 mg keratan sulfate—0.002 units of keratanase II. The digest is done in 10 mM s odium acetate pH 6.8, at 37 C for 30 hours, with no neuraminidase inhibitor present. Following this digestion the enzyme is inactivated by boiling the sample for 2 minutes.

It is possible to use enzymes to differentiate between choindroitin sulfate and dermatan sulfate. Chondroitinase AC I and II cleave only at glucuronate-containing disaccharides i.e. chondroitin sulfate, while chondroitinase B cleaves only at iduronate-containing disaccharides i.e. dermatan sulfate. Chondroitinase ABC cleaves either.

Chondroitin ABC Lyase (Proteus vulgaris, EC 4.2.2.4)

Digestion buffer 0.1 M TRIS/HCl pH 8.0; [optionally add 1 mM NaF (to inhibit sulfatases)]; for digesting glycosaminoglycans to completion 0.3 units/mg glycosaminoglycan is applied at 37 C.

While certain representative embodiments and details have been shown for purposes of illustrating the invention, it will be apparent to those skilled in the art that various changes in the methods and apparatus disclosed herein may be made without departing from the scope of the invention, which is defined in the appended claims.

1. A glycosaminoglycan measuring device, comprising:
a sample entry port adapted to accept a bodily fluid sample;
a glycosaminoglycan separation cartridge coupled to the sample entry port and adapted to separate glycosaminoglycans from interfering substances in the sample; and
a detection apparatus comprising a detection chamber coupled to the glycosaminoglycan separation cartridge and adapted to detect the separated glycosaminoglycans.

2. The glycosaminoglycan measuring device of claim 1, further including a reagent storage device coupled to the glycosaminogiycan separation cartridge and adapted to store reagents for delivery to the glycosaminoglycan separation cartridge.

3. The glycosaminoglycan measuring device of claim 2, further including a first pump coupled to the glycosaminoglycan separation cartridge and adapted to pump fluids into the separation cartridge.

4. The glycosaminoglycan measuring device of claim 3, further including a second pump coupled to the detection apparatus and adapted to pump fluids to the detection chamber.

5. The glycosaminoglycan measuring device of claim 4, further including a controller adapted to control the operation of the first and second pumps.

6. The glycosaminoglycan measuring device of claim 5, wherein the controller includes a processor, a computer readable memory, and a routine stored on the computer readable memory and adapted to be executed on the processor to control the operation of the first and second pumps.

7. The glycosaminoglycan measuring device of claim 1, further including a user interface device operably connected to the detection apparatus, the user interface device including a user interface, a processor, a computer readable memory and a routine stored on the computer readable memory and adapted to be executed on the processor to analyze the output of the detection apparatus and display results of analysis to a user via the user interface.

8. The glycosaminoglycan measuring device of claim 7, wherein the user interface is adapted to prompt a user to log in to the device, initialize the device, enter patient information, prime the device, start the routine, clean the device, reset a cycle counter, enter comments, enter known standard solutions, calculate standard curves, calculate amounts of glycosaminoglycans based on a standard curve, record user actions, or shutdown the device.

9. The glycosaminoglycan measuring device of claim 1, wherein the glycosaminoglycan separation cartridge comprises an ion exchange resin.

10. The glycosaminoglycan measuring device of claim 1, wherein the detection apparatus is a spectrophotometer.

11. The glycosaminoglycan measuring device of claim 1, further comprising a temperature controller operably connected to the detection apparatus.

12. The glycosaminoglycan measuring device of claim 11, wherein the temperature controller is a thermofoil heater.

13. The glycosaminoglycan measuring device of claim 1, further comprising:
a first fluid passageway coupled between the sample entry port and the glycosaminoglycan separation cartridge and adapted to deliver the sample from the sample entry port to the glycosaminoglycan separation cartridge; and
a second fluid passageway coupled between the glycosaminoglycan separation cartridge and the detection chamber and adapted to deliver the separated glycosaminoglycans from the glycosaminoglycan separation cartridge to the detection chamber.

14. The glycosaminoglycan measuring device of claim 13, further comprising one or more valves adapted to control liquid flow in one or more of the fluid passageways.

15. A glycosaminoglycan measuring device comprising:
a sample entry port coupled to a first transport line;
a first reagent container coupled to a second transport line;
a second reagent container coupled to a third transport line;
a glycosaminoglycan separation cartridge coupled to one or more transport lines;
a waste discharging port coupled to a fourth transport line;
a detecting reagent container coupled to a fifth transport line;
a pump operably connected to one or more of the transport lines; and
a detection apparatus operably connected to the device.

16. A device according to claim 15, further comprising a reagent measuring loop in communication with one or more transport lines.

17. A device according to claim 15, further comprising a gas entry port coupled to one or more transport lines.

18. A device according to claim 15, further comprising a cleaning solution container coupled to a sixth transport line.

19. A device according to claim 15, further comprising a transport line adapted to mix liquids.

20. A device according to claim 15, wherein the glycosaminoglycan separation cartridge comprises an ion exchange resin.

21. A device according to claim 15, wherein the detection apparatus is a spectrophotometer.

22. A device according to claim 15, further comprising a temperature controller operably connected to the detection apparatus.

23. A device according to claim 15, further comprising a valve coupled to one or more transport lines and adapted to direct liquid flow.

24. A glycosaminoglycan measuring device comprising a sample entry port, separator means for substantially separating glycosaminoglycans from a sample delivered at the sample entry port to produce separated glycosaminoglycans, and detection means for detecting the separated glycosaminoglycans.

25. A method of measuring glycosaminoglycans in a body fluid sample, comprising the steps of:
(a) automatically delivering a portion of the sample to a glycosaminoglycan separation cartridge;
(b) separating glycosaminoglycans from interfering substances using the glycosaminoglycan separation cartridge;
(c) automatically delivering the separated glycosaminoglycans to a detection apparatus;
(d) combining a detection reagent with the separated glycosaminoglycans; and
(e) detecting the amount of separated glycosaminoglycans by detecting the amount of detection reagent bound to the separated glycosaminoglycans.

26. The method of claim 25, further comprising the step of analyzing an output of the detection apparatus and displaying the analyzed output to a user.

27. The method of claim 25, wherein automatic delivery occurs in response to user input.

28. A method according to claim 25, wherein step (b) comprises the steps of:
binding glycosaminoglycans to a solid phase;
removing interfering substances; and
eluting glycosaminoglycans from the solid phase.

29. A method according to claim 28, wherein the removing step is contacting the solid phase with a buffered solution comprising Na+ at a concentration ranging from 350 mM to 450 mM.

30. A method according to claim 28, wherein the eluting step is contacting the solid phase with a buffered solution comprising Na+ at a concentration ranging from 700 mM to 1500 mM.

31. A method according to claim 25, wherein the body fluid sample is selected from the group consisting of whole blood, plasma, serum, urine, cerebrospinal fluid, pleural fluid, extracts of tissue biopsies, saliva, semen, stool, sputum, tears, and mucus.

32. A method according to claim 25, wherein the body fluid sample is blood or urine.

33. A method according to claim 25, wherein the separated glycosaminoglycans include glycosaminoglycans selected from the group consisting of chondroitin sulfates, dermatan sulfates, keratan sulfates, heparan sulfates, heparin sulfates, and heparin.

34. A method according to claim 25, wherein the separated glycosaminoglycans include heparin.

35. A method according to claim 25, wherein the glycosaminoglycan capturing cartridge includes an ion exchange resin.

36. A method according to claim 25 wherein the detection reagent is a metachromic dye that specifically binds glycosaminoglycans.

37. A method according to claim 25, wherein the detection reagent is selected from the group consisting of alcian blue, azure A, azure B, dimethylmethylene blue, fuchsin, acridine orange, proflavine, neutral red, and brilliant cresyl blue.

38. A method according to claim 25, wherein the detection reagent is dimethylmethylene blue (DMMB).

39. A method according to claim 25, wherein the detection apparatus is a spectrophotometer.

40. A method according to claim 25, further comprising the use of software designed to quantify the amount of glycosaminoglycans present based on detecting the detection reagent bound to the separated glycosaminoglycans.

41. A method according to claim 25, wherein step (e) occurs at a regulated temperature.

42. The method according to claim 25, further comprising the step of adding one or more glycosaminoglycan-specific degrading enzymes to, the sample before step (a).

43. A method according to claim 42, wherein the glycosaminoglycan-specific degrading enzyme is selected from the group consisting of chondroitinase B, chondroitinase AB, heparinase, heparitanase, heparitanase I, heparitanase II, keratanase, α-L-iduronidase, iduronate sulfatase, Heparan N-sulfatase, N-acetylglucosaminidase, α-glucosamine-N-acetyltransferase, α-glucosamine-6-sulfatase, N-acetylgalactosamine-6-sulfatase, B-galactosidase, N-acetylgalactosamine-4-sulfatase, and B-glucuronidase.

44. A method according to claim 43, wherein the glycosaminoglycan-specific degrading enzyme is chondroitinase B.

45. A method according to claim 25, wherein step (e) comprises the step of shining light at a wavelength of about 526 nm+/−5 nm at the separated glycosaminoglycans.

46. A method according to claim 25, wherein step (e) comprises the step of shining light at a wavelength of about 592 nm+/−5 nm at the separated glycosaminoglycans.

47. A method according to claim 25, performed substantially concurrent with heparin therapy.

48. A method according to claim 25, performed for the purpose of monitoring heparin therapy.

49. A method according to claim 25, performed for the purpose of measuring endogenous heparin levels.

50. A method according to claim 25, performed for the purpose of screening newborns, infants, or children for disorders associated with abnormal glycosaminoglycan concentration levels.

51. A method according to claim 25, performed for the purpose of diagnosing mucopolysaccharidoses.

52. A method of using the device of claim 1 for monitoring the amount of heparin in a patient receiving heparin therapy.

53. A method of using the device of claim 1 for measuring endogenous heparin levels.

54. A method of using the device of claim 1 for the purpose of screening newborns, infants, or children for disorders associated with abnormal glycosaminoglycan concentration levels.

55. A method of using the device of claim 1 for the purpose of diagnosing mucopolysaccharidoses.

56. A kit comprising instructions for using a device according to claim 1 and one or more reagents for use in a device according to claim 1.

57. A kit comprising instructions for using a device according to claim 1 and one or more standards of known glycosaminoglycan concentration.
